(19) United States
(12) Patent Application Publication
(10) Pub. No.: US 2025/0115871 A1
(43) Pub. Date: Apr. 10, 2025

(54) METHODS TO IMPROVE ENDURING HEMATOPOIETIC STEM CELL TRANSPLANTATION

(71) Applicant: Trailhead Biosystems Inc., Beachwood, OH (US)

(72) Inventor: Angelica M. GOMES UELTSCHY, Shaker Heights, OH (US)

(21) Appl. No.: 18/910,889

(22) Filed: Oct. 9, 2024

Related U.S. Application Data
(60) Provisional application No. 63/564,287, filed on Mar. 12, 2024.
(60) Provisional application No. 63/543,149, filed on Oct. 9, 2023.

(51) Int. Cl. C12N 5/0789; A61K 35/28

(52) U.S. Cl. CPC C12N 5/0647; A61K 35/28; C12N 2500/36; C12N 2501/16; C12N 2501/73

(57) ABSTRACT

Methods for enhancing engraftment of hematopoictic stem cells (HSCs) (e.g., human CD34+HSCs) in a subject are provided in which the HSCs are cultured prior to transplantation under conditions that enhance engrafiment following transplantation.

### RELATED APPLICATIONS

This application claims priority to U.S. Provisional Application No. 63/543,149, filed Oct. 9, 2023, and U.S. Provisional Application No. 63/564,287, filed Mar. 12, 2024, the entire contents of each of which is hereby incorporated by reference.

### BACKGROUND OF THE INVENTION

Hematopoietic stem cells (HSCs) are rare cells naturally found in human bone marrow and umbilical cord blood, and even more rarely in peripheral blood. HSCs are typically defined by the expression, or lack of expression, of particular markers, such as an HSC population characterized by expression of CD34 and lack of expression of Lineage-specific markers and CD38 (Lin-CD34+CD38-). In addition, there is evidence for a CD34-population in umbilical cord blood that is thought to signal a different category of HSC (see e.g., Sonoda (2021) Experimental Hematology 96:13-26). HSC behavior is plastic, which means upon receiving signals from the environment, HSCs can exit the quiescent state and undergo differentiation. The classical model of hematopoiesis is characterized by HSCs giving rise to multipotent progenitors, which can differentiate into more committed progenitors and eventually into fully mature hematopoietic cells.

Hematopoietic stem cell transplantation (HSCT) is characterized by intravenous infusion of hematopoietic stem and progenitor cells for generation of blood cells. HSCT is reviewed in, for example, Passweg et al. (2020) Bone Marrow Transplant. 55:1604-1613. HSCs used for transplants have been obtained from bone marrow, cord blood and peripheral blood. HSCs that are derived from the patient that is receiving the transplant are characterized as autologous transplants. If the transplant is conducted using cells from a matched donor, the transplant is characterized as an allogeneic transplant. The main indications for HSCT are myeloid cancer, lymphoid malignancies, solid tumors, and non-malignancies disorders such as aplastic anemia.

For allogeneic HSCT, in approximately 60-70% of cases, peripheral blood HSCs are used. Because of the low HSC number in blood, prior to collection mobilizing agents are used to increase the concentration of circulating HSC and progenitors. In both allogeneic and autologous settings, peripheral blood HSCs have been associated with better patient outcomes, such as decreased relapse rate and improvement in overall disease-free survival, as compared to bone marrow cells (see e.g., J. Clin. Oncol. (2005) 23:5074-5087). However, HSCT using peripheral blood HSC has been associated with the risk of developing chronic graft versus host disease

(GVHD) in patients. During GVHD, donor cells initiate an immunological response against antigens in the patient's tissue, which can lead to pathology of normal organs. There are many factors that impact GVHD occurrence, such as human leukocyte antigen (HLA) disparity. HLA are cell surface proteins involved in immune recognition. HLA allows the immune system to recognize what is self and what is foreigner. The HLA system is complex and, considering the many different possible combinations and different types of HLA alleles, it can take a long time to identify a matching donor.

HSCT using cord blood cells has advantages in this aspect, because of lower incidence of graft-versus host disease (immature less reactive T-cells), and allowance for greater HLA mismatch (see e.g., Ruggeri et al. (2016) HLA: Immune Resp. Genet. 87:413-421). However, again limited amounts of available cord blood HSCs limit this approach.

Accordingly, efforts have focused on culture systems for expanding CD34+HSCs that can be applied to cord blood as well as other sources of CD34+HSCs. Various culture systems for expanding CD34+HSCs have been described, typically including a culture medium supplemented with stem cell factor (SCF) and thrombopoietin (TPO), along with other cytokines and small molecules. For example, Nishino et al. report expanding HSCs by culturing for seven days in a medium containing SCF, TPO and garcinol, a potent inhibitor of histone acetyltransferase (Nishino et al. (2011) PLOS One 6:024298). Himburg et al. report expanding HSCs by culturing in media containing SCF, TPO, Flt-3 ligand and pleiotrophin, the latter of which activates PI3K signaling (Himburg et al. (2010) Nat. Med. 16:475-482). CD34+HSCs from umbilical cord blood have been expanded ex vivo by culture in a base media containing SCF, TPO, Flt-3 ligand and low density lipoproteins to which was added an inhibitor of the JNK pathway (Xiao et al. (2019) Cell. Discovery 5:2). Alternatively, CD34+HSCs from umbilical cord blood were expanded ex vivo by culture in a media containing SCF, TPO, Flt-3 and IL-3 for 16 hours followed by further culture for seven days with the deacetylase inhibitor valproic acid (VPA) (Papa et al. (2019) J. Vis. Exp. DOI: 10.3791/59532).

While certain advances have been made, additional approaches and methods are still needed for improving the use of CD34+HSCs, as well other subpopulations of HSCs such as CD34-HSCs, in HSCT, in particular approaches that enhance engraftment of the HSCs. This would potentially decrease the cell number needed during HSCT, unlocking the potential of cord blood units with lower HSC content. Furthermore, to enable these discoveries, approaches to study HSCT techniques in preclinical settings are needed.

### SUMMARY OF THE INVENTION

This disclosure provides methods of enhancing engraftment of hematopoietic stem cells (HSCs) (e.g., human CD34+ and CD34-HSCs or HSCs from other species, such as rodents or primates, as described further herein) in a subject, whether a human, animal, or humanized/chimerized animal modified with human features, for example such that long term endurance of the HSC graft is improved in the subject. The approach and methods described herein provide conditions for culture of the HSCs prior to transplantation into a subject that result in enhanced engraftment of the HSCs in the subject following transplantation (as compared to engraftment using control pretreatments or no pretreatment). This enhanced engraftment includes increased levels of differentiated hematopoietic cells derived from the graft in peripheral blood, bone marrow and spleen of the graft recipient, indicating that the culture conditions provided herein allow for greater repopulation of the hematopoietic system (as compared to the control treatments or no pretreatment) (see Examples 3-4). Moreover, this enhanced engraftment capacity of the culture conditions of the disclosure was shown to have a long-term enduring effect through secondary transplant studies (see Example 5).

The compositions and methods of the disclosure are suitable for use with human CD34+ and CD34-HSCs, for example to improve HSC engraftment in a human subject or in humanized/chimerized animal models. Moreover, the compositions and methods can be used with non-human HSCs into non-human subjects. Sources of HSCs are described further herein.

Although not intending to be limited by mechanism, the methods of the disclosure are based on culture conditions that enhance the conversion of HSCs to long term hematopoietic stem cells (LT-HSCs). Whereas prior approaches have focused on culture conditions for expansion of HSCs in culture, the ability of the methods of the disclosure to enhance conversion to LT-HSCs is advantageous, since these cells are at the top of the hematopoietic differentiation tree and possess self-renewal and multilineage competence. Another advantage of the approach of the disclosure is that the culture conditions avoid the use of stem cell factor (SCF) and thrombopoietin (TPO) typically used in other protocols, as well as lacking serum or other exogenous cytokines. Furthermore, the methods of the disclosure can readily be applied in a clinical setting, such as in the use of HSCT for the treatment of a variety of hematological disorders such as myeloid cancer, lymphoid malignancies, solid tumors, and non-malignancies disorders such as aplastic anemia.

Accordingly, in one aspect, the disclosure pertains to a method of preparing hematopoietic stem cells (HSCs) for transplantation into a subject, the method comprising:
culturing HSCs in a culture media comprising a TGFß agonist, a bioactive phospholipid, an aryl hydrocarbon receptor (AhR) agonist and a histone deacetylase (HDAC) inhibitor for 12-36 hours (e.g., 24 hours), wherein the culture media lacks stem cell factor (SCF) and thrombopoietin (TPO); and
administering the HSCs into the subject.

In embodiments, the HSCs are human HSCS, which may be CD34+ or CD34-. In embodiments, the HSCs are non-human HSCs, which may be CD34+, CD34-, or CD150+CD41-CD34-Kit+Sca-1+Lineage-. In embodiments, CD34+ or CD34-HSCs (e.g., human CD34+ or CD34-HSCs) are administered to a human subject. In embodiments, CD34+, CD34-, or CD150+CD41-CD34-Kit+Sca-1+Lineage-HSCs (e.g., human or non-human HSCs) are administered to a non-human subject, such as a non-human animal or humanized/chimerized animal that serves as a model for a human or animal condition (e.g., human or veterinary disease or disorder).

In embodiments, the cells are cultured for 18-32 hours prior to administration. In embodiments, the cells are cultured for about 24 hours prior to administration. In embodiments, the cells are cultured for 24 hours prior to administration.

In embodiments, culturing the HSCs enhances engraftment of the HSCs in the subject as compared to absence of culturing prior to administration. In embodiments, enhanced engraftment comprises increased engraftment of the HSCs in the peripheral blood. In embodiments, enhanced engraftment comprises increased engraftment of the HSCs in the spleen and/or bone marrow.

In an embodiment, the culture media consists essentially of a basal media, a TGFß agonist, a bioactive phospholipid, an AhR agonist and an HDAC inhibitor.

In an embodiment, the TGFB agonist is Activin A. In an embodiment, Activin A is present in the culture media in a concentration range of 15-25 ng/ml. In an embodiment, Activin A is present in the culture media at a concentration of 20 ng/ml. Other concentrations and ranges, as well as other suitable TGFß agonists, are disclosed herein.

In an embodiment, the bioactive phospholipid is lysophosphatidic acid (LPA). In an embodiment, LPA is present in the culture media in a concentration range of 150-250 nM. In an embodiment, LPA is present in the culture media at a concentration of 200 nM. Other concentrations and ranges, as well as other suitable bioactive phospholipids, are disclosed herein.

In an embodiment, the AhR agonist is 6-Formylindolo [3,2-b] carbazole (FICZ). In an embodiment, FICZ is present in the culture media in a concentration range of 400-600 nM. In an embodiment, FICZ is present in the culture media at a concentration of 500 nM. Other concentrations and ranges, as well as other suitable AhR agonists, are disclosed herein.

In an embodiment, the HDAC inhibitor is valproic acid (VPA). In an embodiment, VPA is present in the culture media in a concentration range of 100-200 μM. In an embodiment, VPA is present in the culture media at a concentration of 150 μM. Other concentrations and ranges, as well as other suitable HDAC inhibitors, are disclosed herein.

In an embodiment, the TGFB agonist is Activin A, bioactive phospholipid is LPA, the AhR agonist is FICZ and the HDAC inhibitor is VPA. In an embodiment, Activin A is present in a concentration range of 15-25 ng/ml, LPA is present in a concentration range of 150-250 nM, FICZ is present in a concentration range of 400-600 nM and VPA is present in a concentration range of 100-200 μM.

In an embodiment, Activin A is present at a concentration of 20 ng/ml, LPA is present at a concentration of 200 nM, FICZ is present at a concentration of 500 nM and VPA is present at a concentration of 150 μM.

In an embodiment wherein the culture media further comprises an antioxidant and a Notch agonist, the antioxidant can be, for example, Vitamin C and the Notch agonist can be, for example, Yhhu 3792. In an embodiment, Vitamin C is present in a concentration range of 50-150 μM (e.g., at a concentration of 100 μM). In an embodiment, Yhhu 3792 is present in a concentration range of 500-1000 nM (e.g., at a concentration of 750 nM). Other concentrations and ranges, as well as other suitable antioxidants and Notch agonists, are disclosed herein.

In another embodiment, the disclosure pertains to a method of enhancing engraftment of hematopoietic stem cells (HSCs), which may be, for example, human and either CD34+ or CD34-, or may be primate and either CD34+ or CD34-, or may be rodent and CD150+CD41-CD34-Kit+Sca-1+Lineage-, in a subject, the method comprising:
culturing HSCs in a culture media consisting essentially of a basal media, a TGFß agonist, a bioactive phospholipid, an aryl hydrocarbon receptor (AhR) agonist and a histone deacetylase (HDAC) inhibitor for 12-36 hours (e.g., 24 hours); and
transplanting the HSCs into the subject,
wherein engraftment is enhanced compared to not culturing the HSCs prior to transplanting.

In embodiments, the CD34+ or CD34-HSCs are human HSCS. In embodiments, the CD34+ or CD34-HSCs or CD150+CD41-CD34-Kit+Sca-1+Lineage-HSCs are non-human HSCs. In embodiments, CD34+ or CD34-HSCs (e.g., human CD34+ or CD34-HSCs) are administered to a human subject. In embodiments, CD34+ or CD34-or

CD150+CD41-CD34-Kit+Sca-1+Lineage-HSCs (e.g., human or non-human HSCs) are administered to a non-human or humanized/chimerized subject, such as a non-human animal that serves as a model for a human or animal condition (e.g., human or veterinary disease or disorder).

In embodiments, the cells are cultured for 18-32 hours prior to administration. In embodiments, the cells are cultured for about 24 hours prior to administration. In embodiments, the cells are cultured for 24 hours prior to administration.

In embodiments, enhanced engraftment comprises increased engraftment of the HSCs in the peripheral blood. In embodiments, enhanced engraftment comprises increased engraftment of the HSCs in the spleen and/or bone marrow.

In an embodiment, the TGFB agonist is Activin A, the bioactive phospholipid is LPA, the AhR agonist is FICZ and the HDAC inhibitor is VPA.

In an embodiment, Activin A is present in a concentration range of 15-25 ng/ml, LPA is present at in a concentration range of 150-250 nM, FICZ is present in a concentration range of 400-600 nM and VPA is present in a concentration range of 100-200 μM.

In an embodiment, Activin A is present at a concentration of 20 ng/ml, LPA is present at a concentration of 200 nM, FICZ is present at a concentration of 500 nM and VPA is present at a concentration of 150 μM.

Other concentrations and ranges, as well as other suitable TGFß agonists, bioactive phospholipids, AhR agonists and HDAC inhibitors are disclosed herein.

The methods of the disclosure can be carried out with any suitable source of CD34+, CD34-, or CD150+CD41-CD34-Kit+Sca-1+Lineage-HSCs. In an embodiment, the HSCs are from umbilical cord blood. In an embodiment, the HSCs are from bone marrow. In an embodiment, the HSCs are from peripheral blood.

Other features and advantages of the invention will be apparent from the following detailed description and claims.

### BRIEF DESCRIPTION OF THE DRAWINGS

FIGS. 1A-1B are graphs showing results from flow cytometry analyses of cord blood CD34 cells grown for 24h in indicated LT-HSC culture media or control media (SCF and TPO) or uncultured. FIG. 1A shows results for CD34/CD38 staining. FIG. 1B shows the results for CD45RA/CD90 staining gated from CD34+ and CD38-population.

FIGS. 2A-2B are graphs showing results for analyses of human LT-HSC engraftment in peripheral blood of NSG mice. FIG. 2A shows results for human cell engraftment as measured by human CD45 expression, presented as the percentage of total CD45 cells in the peripheral blood of NSG mice at 11, 13 and 19 weeks post-transplantation and summarized over time (left to right). FIG. 2B shows results presented as the percentage of hCD19+ cells gated from hCD45+ cells at 11, 13 and 19 weeks in peripheral blood (left to right).

FIGS. 3A-3B are graphs showing results for analyses of human LT-HSC engraftment in spleen and bone marrow of NSG mice. FIG. 3A shows results for human cell engraftment as measured by human CD45 expression, presented as the percentage of total CD45 cells in the spleen and bone marrow of NSG mice at 19 weeks post-transplantation. FIG. 3B shows results presented as the percentage of hCD19+, CD14+ and CD3+ cells (left to right) gated from hCD45+ cells at 19 weeks in spleen samples.

FIGS. 4A-4B are graphs showing results for analyses of human cell engraftment in peripheral blood in a secondary transplant model. FIG. 4A shows results for human cell engraftment as measured by human CD45 expression, presented as the percentage of total CD45 cells in the peripheral blood of NSG mice at 20 weeks post-transplantation in a secondary transplant model, human cell engraftment over time and area under the curve from time 0 to 20 weeks of secondary transplantation (left to right). FIG. 4B shows results presented as the percentage of hCD19+ cells gated from hCD45+ cells at 20 weeks in peripheral blood samples.

FIG. 5 shows results for analyses of human cell engraftment in spleen and bone marrow in a secondary transplant model. Human cell engraftment as measured by human CD45 expression, presented as the percentage of total CD45 cells in the spleen (left) and bone marrow (right) of NSG mice 20 weeks post-transplantation in a secondary transplant model.

### DETAILED DESCRIPTION OF THE INVENTION

Described herein are approaches and methodologies for improving the endurance of an HSC graft (e.g., a human, rodent, or primate HSC graft) in a subject that results in enhanced engraftment and greater repopulation as compared to prior methods, such as those that utilize culture in a media that comprises SCF and TPO. The compositions and methods described herein are suitable for use with human HSCs in humans, such as to improve HSC engraftment in a human subject. Furthermore, the compositions and methods can be used with human HSCs in non-human or humanized/chimerized systems (e.g., in animal models, such as for pre-clinical research). Moreover, the compositions and methods can be used with non-human HSCs in non-human systems, such as for research or clinical purposes, as described further herein.

Various aspects of the invention are described in further detail in the following subsections.

### I. Conversion Approach and Engraftment Enhancement

With regard to the success of HSCT, it is known that the cell dose is critical for hematopoietic engraftment. While there has been some progress in strategies for HSC expansion in culture, the limited number of HSCs that are found in a cord blood unit is still a challenge for the broad use of cord blood units for transplantations. For example, Barker et al. (Blood Adv. (2019) 3:1267-1271) analyzed total nuclear counts and CD34+content in cord blood units in 20 public cord blood banks available in America. Then, they analyzed the number of units that meet the cell dose criteria for a single-unit graft by patient body weight. (TNCs, ≥2.5×107/kg; CD34+ cells, >1.5×105/kg per current guidelines7.9). Half of the units analyzed met the minimum criteria for patients that weigh 30 Kg. However, for an average adult patient weighing 70 Kg just 4% of cord blood units available would be adequate, having enough total nuclear cells as well as CD34+ cells.

Thus, methods to improve engraftment of HSC are crucial to increase the number of cord blood units that are usable in cord blood banks. This also is of relevance in the context of increasing chances of a better HLA-matching between available cord blood units and patients.

Prior attempts to improve engraftment of CD34+ from cord blood have focused on the use of cytokines for expansion of cell number. Such approaches, referred to herein as “expansion methods”, are based on the use of various hematopoietic cytokines in culture, may or may not be serum-free, and usually take more than 24 hours prior to transplantation. Depending on the method of expansion, generation of more total cell numbers at the expense of HSCs has been observed. This is mainly explained by the fact that some cytokines and culture conditions will instruct lineage choices.

In contrast, the approach of the disclosure is based on short term culture (e.g., 12-36 hours, such as 24 hours) of the HSCs prior to transplantation in a culture media that does not use hematopoietic cytokines or serum (e.g., lacks SCF and TPO), and is not based on expansion. Rather the culture media is based on conversion, wherein the media maintains and promotes (enhances, stimulates) long-term HSC (LT-HSCs) phenotype. The culture media used for conversion was first developed with the goal to optimize expression of genes associated with the stem cell state in HSCs, such as CHRBP, Mecom, Meg3, HOPX, LMO2, CD34. Certain aspects of the HSC culture media have been described in US Patent Publication US2023/0167408, as well as PCT Publication WO 2023/101943, the contents of each of which are hereby incorporated in their entirety. However, use of the culture media in the context described herein for enhancement of HSC engraftment under specified conditions has not been described.

As described in detail in the Examples, the culture conditions of the disclosure (described further in subsection II) enhance engraftment of CD34+HSCs in a subject. Thus, the methods of the disclosure promote enduring HSC transplantation and repopulation of cells of the hematopoietic system in the recipient. Example 1 provides a culture media, set forth in Table 1 and referred to herein as LT-HSCOPT, that increases expression of LT-HSC markers on CD34+HSCs after only 24 hours of culture (see Example 2). Culture of CD34+HSCs in this media for 24 hours prior to transplantation into a subject led to enhanced engraftment in the subject, as compared to controls or untreated cells (see Examples 3-4). Moreover, secondary transplant studies demonstrated the ability of the culture protocol to lead to enduring engraftment and repopulation of the hematopoietic system (see Example 5). The methods of the disclosure are described in further detail in subsection II below.

### II. Methods of Preparing Cells for Enhancing Engraftment

In one aspect, the disclosure pertains to a method of preparing hematopoietic stem cells (HSCs) (e.g., human or primate CD34+ or CD34-HSCs or rodent CD150+CD41-CD34-Kit+Sca-1+Lineage-HSCs) for transplantation into a subject, the method comprising:
culturing HSCs in a culture media comprising a TGFß agonist, a bioactive phospholipid, an aryl hydrocarbon receptor (AhR) agonist and a histone deacetylase (HDAC) inhibitor for 12-36 hours, wherein the culture media lacks stem cell factor (SCF) and thrombopoietin (TPO); and
administering the HSCs into the subject.

In embodiments, the HSCs are human. In other embodiments, the HSCs are non-human (e.g., rodent or primate HSCs). Different HSCs and sources thereof are described further in subsection III below.

In embodiments, the cells are cultured in the culture media for 18-32 hours, 20-30 hours, 22-26 hours, about 24 hours or 24 hours.

In embodiments, culturing the HSCs (e.g., human or primate CD34+ or CD34-HSCs or rodent CD150+CD41-CD34-Kit+Sca-1+Lineage-HSCs) enhances engraftment of the HSCs in the subject as compared to absence of culturing prior to administration. In embodiments, enhanced engraftment comprises increased engraftment of the HSCs in the peripheral blood. In embodiments, enhanced engraftment comprises increased engraftment of the HSCs in the spleen and/or bone marrow.

In an embodiment, the culture media consists essentially of a basal media, a TGFB agonist, a bioactive phospholipid, an AhR agonist and an HDAC inhibitor.

In an embodiment, the TGFß agonist is Activin A. In an embodiment, Activin A is present in the culture media in a concentration range of 15-25 ng/ml. In an embodiment, Activin A is present in the culture media at a concentration of 20 ng/ml. Other concentrations and ranges, as well as other suitable TGFB agonists, are described further in subsection IV below.

In an embodiment, the bioactive phospholipid is lysophosphatidic acid (LPA). In an embodiment, LPA is present in the culture media in a concentration range of 150-250 nM. In an embodiment, LPA is present in the culture media at a concentration of 200 nM. Other concentrations and ranges, as well as other suitable bioactive phospholipids, are described further in subsection IV below.

In an embodiment, the AhR agonist is 6-Formylindolo [3,2-b] carbazole (FICZ). In an embodiment, FICZ is present in the culture media in a concentration range of 400-600 nM. In an embodiment, FICZ is present in the culture media at a concentration of 500 nM. Other concentrations and ranges, as well as other suitable AhR agonists, are described further in subsection IV below.

In an embodiment, the HDAC inhibitor is valproic acid (VPA). In an embodiment, VPA is present in the culture media in a concentration range of 100-200 μM. In an embodiment, VPA is present in the culture media at a concentration of 150 μM. Other concentrations and ranges, as well as other suitable HDAC inhibitors, are described further in subsection IV below.

In an embodiment, the TGFB agonist is Activin A, bioactive phospholipid is LPA, the AhR agonist is FICZ and the HDAC inhibitor is VPA. In an embodiment, Activin A is present in a concentration range of 15-25 ng/ml, LPA is present in a concentration range of 150-250 nM, FICZ is present in a concentration range of 400-600 nM and VPA is present in a concentration range of 100-200 μM. In an embodiment, Activin A is present at a concentration of 20 ng/ml, LPA is present at a concentration of 200 nM, FICZ is present at a concentration of 500 nM and VPA is present at a concentration of 150 μM. Other concentrations and ranges, as well as other suitable TGFB agonists, bioactive phospholipids, AhR agonists, and HDAC inhibitors, are described further in subsection IV below.

In an embodiment wherein the culture media further comprises an antioxidant and a Notch agonist, the antioxidant can be, for example, Vitamin C and the Notch agonist can be, for example, Yhhu 3792. In an embodiment, Vitamin C is present in a concentration range of 50-150 μM (e.g., at a concentration of 100 μM). In an embodiment, Yhhu 3792 is present in a concentration range of 500-1000 nM (e.g., at a concentration of 750 nM). Other concentrations and ranges, as well as other suitable antioxidants and Notch agonists, are described further in subsection IV below.

In another embodiment, the disclosure pertains to a method of enhancing engraftment of hematopoietic stem cells (HSCs) (e.g., human or primate CD34+ or CD34-HSCs or rodent CD150+CD41-CD34-Kit+Sca-1+Lineage-HSCs HSCs) in a subject, the method comprising: culturing HSCs in a culture media consisting essentially of a basal media, a TGFß agonist, a bioactive phospholipid, an aryl hydrocarbon receptor (AhR) agonist and a histone deacetylase (HDAC) inhibitor for 12-36 hours; and transplanting the HSCs into the subject, wherein engraftment is enhanced compared to not culturing the HSCs prior to transplanting.

In embodiments, the CD34+ or CD34-HSCs are human. In other embodiments, the CD34+, CD34-, or CD150+CD41-CD34-Kit+Sca-1+Lineage-HSCs are non-human (e.g., rodent or primate HSCs). Different HSCs and sources thereof are described further in subsection III below.

In embodiments, the cells are cultured in the culture media for 18-32 hours, 20-30 hours, 22-26 hours, about 24 hours or 24 hours.

In embodiments, enhanced engraftment comprises increased engraftment of the HSCs in the peripheral blood. In embodiments, enhanced engraftment comprises increased engraftment of the HSCs in the spleen and/or bone marrow.

In an embodiment, the TGFß agonist is Activin A, the bioactive phospholipid is LPA, the AhR agonist is FICZ and the HDAC inhibitor is VPA.

In an embodiment, Activin A is present in a concentration range of 15-25 ng/ml, LPA is present at in a concentration range of 150-250 nM, FICZ is present in a concentration range of 400-600 nM and VPA is present in a concentration range of 100-200 μM.

In an embodiment, Activin A is present at a concentration of 20 ng/ml, LPA is present at a concentration of 200 nM, FICZ is present at a concentration of 500 nM and VPA is present at a concentration of 150 μM.

Other concentrations and ranges, as well as other suitable TGFß agonists, bioactive phospholipids, AhR agonists and HDAC inhibitors, are described further in subsection IV below.

In combination with the chemically-defined culture media described herein, the methods of the disclosure utilize standard culture conditions established in the art for cell culture. For example, cells can be cultured at 37° C. and under 5% CO2 conditions. Cells can be cultured in standard culture vessels or plates, such as culture dishes, culture flasks or 96-well plates.

A basal media typically is used as the starting media to which supplemental agents are added. For example, in an embodiment, the commercially available StemSpan™ SFEM II media (STEMCELL Technologies) is used as basal media.

Approaches for assessing engraftment of the HSCs are established in the art, including but not limited to the flow cytometry approaches (e.g., as described in detail in the Examples) as well as other established methods for measuring cell numbers, cell counts or cell levels. In embodiments, increased engraftment of the HSCs in the peripheral blood comprises an increased number of cells in the peripheral blood that are derived from the donor HSCs (e.g., an increased percentage of donor-derived cells in peripheral blood). In embodiments, enhanced engraftment comprises increased engraftment of the HSCs in the spleen or bone marrow comprises an increased number of cells in the spleen or bone marrow that are derived from the donor HSCs (e.g., an increased percentage of donor-derived cells in spleen or bone marrow).

Suitable cells for use in the methods of the disclosure are described in detail in subsection III below.

### III. Cells

The starting cells used in the methods of the disclosure are hematopoietic stem cells (HSCs), such as CD34+, CD34-, or CD150+CD41-CD34-Kit+Sca-1+Lineage-hematopoietic stem cells. In embodiments, the CD34+ or CD34-HSCs are human HSCs. In embodiments, the CD34+, CD34-, or CD150+CD41-CD34-Kit+Sca-1+Lineage-HSCs are non-human HSCs. In embodiments, the non-human HSCs are from a vertebrate animal. In embodiments, the vertebrate animal is a mammal. In embodiments, the vertebrate animal is a primate, non-limiting examples of which include chimpanzees, rhesus macaques and cynomolgus macaques. In embodiments, the vertebrate animal is a rodent, non-limiting examples of which include mice and rats. In embodiments, the vertebrate animal is a domesticated animal, non-limiting examples of which include cows, horses, goats, sheep, dogs, cats and chickens.

The methods can be carried out with any suitable source of HSCs that are amenable for culture in vitro and in particular those that are amenable for transplantation in vivo (i.e., use in HSCT). In an embodiment, the HSCs (e.g., human, rodent, or primate HSCs) are from umbilical cord blood. In an embodiment, the HSCs (e.g., human, rodent, or primate HSCs) are from bone marrow. In an embodiment, the HSCs (e.g., human, rodent, or primate HSCs) are from fetal liver. In an embodiment, the HSCs (e.g., human, rodent, or primate HSCs) are from peripheral blood. In an embodiment, the HSCs are autologous to the transplant recipient. In an embodiment, the HSCs are allogeneic to the transplant recipient.

As used herein, the term “hematopoietic stem cell” (abbreviated as HSC) refers to a multipotent, self-renewing stem cell with multilineage differentiation capabilities, being able to develop into all types of blood cells, including lymphoid-lineage cells and myeloid-lineage cells, such as erythrocytes, leukocytes, platelets, and lymphocytes. In addition to these properties, different populations of HSCs have been defined in humans and non-human species based on surface marker expression. For example, CD34 is a transmembrane phosphoglycoprotein that has been established in the art as a surface marker for HSCs in humans as well as certain other animals, such as primates. Accordingly, in embodiments, the HSCs (human or non-human) are CD34+. Additionally, a CD34-population of HSCs has been described in the art in both human and non-humans (e.g., rodents, such as mice) (see e.g., Osawa et al. (1996) Science 273:242-245; Morel et al. (1998) Exp. Hematol. 26:440-448; Pei (1999) Int. J. Hematol. 70:213-215; Engelhardt et al. (2002) Leukemia 16:1603-1608; Sonoda (2008) J. Autoimmunity 30:136-144; Sonoda (2021) Exp. Hematol. 96:13-26). Accordingly, in other embodiments, the HSCs (human or non-human) are CD34-. Still further, an HSC population in rodents (e.g., mice) has been described that is defined as CD150+CD41-CD34-Kit+Sca-1+Lineage-(see e.g., Osawa et al. (1996) Science 273:242-245). Accordingly, in other embodiments, the HSCs are from rodent (e.g., mice) and are CD150+CD41-CD34-Kit+Sca-1+Lineage-.

When the HSCs come from non-human primates, the markers used to define the non-human primate HSC are similar to the markers used to define human HSCs (see e.g., Radtke et al., (2017) Sci Transl Med 9 (414): caan1145).

Human HSCs are readily obtainable from available sources, including human umbilical cord blood and adult bone marrow. HSCs include both long term HSCs (LT-HSCs) and short term HSCs (ST-HSCs). Long term HSCs (LT-HSCs) are HSCs that are found in the bone marrow or cord blood that, through a process of asymmetric cell division, can self-renew to sustain the stem cell pool or differentiate into short-term HSCs (ST-HSCs) or lineage-restricted progenitors that undergo extensive proliferation and differentiation to produce terminally differentiated cells of the blood lineage. It is believed that LT-HSCs are enriched on the fraction of Lin-CD34+CD38-CD45RA-CD90+ cells. LT-HSCs are quiescent and slow to divide in culture, taking up to 80 hours to first cell division (Cheung and Rando (2013) Nat. Rev. Mol. Cell Biol. 14:329-340). In contrast, short term HSCs (ST-HSCs) by definition have limited self-renewal capacity, generally described as giving rise to lymphohematopoiesis for 4-12 weeks before senescence.

In an embodiment, the HSCs express CD34 (CD34+). In an embodiment, the HSCs do not express CD34 (CD34-). In an embodiment, the HSCs lack expression of the marker Lineage (Lin-). In an embodiment, the HSCs lack expression of CD38 (CD38-). In an embodiment, the HSCs lack expression of CD45RA (CD45RA-). In an embodiment, the HSCs express CD90 (CD90+). In an embodiment, the HSCs are Lin-CD34+CD38-CD45RA-CD90+ cells. In an embodiment, the HSCs are CD150+CD41-CD34-Kit+Sca-1+Lineage-.

In embodiments, the HSCs express one or more genes associated with the HSC phenotype (also referred to herein as HSC-associated genetic markers), non-limiting examples of which include CHRBP, Mecom, Meg3, HOPX, LMO2, CD34, TALI and GATA2.

In an embodiment, the LT-HSCs are CD34+. In an embodiment, the LT-HSCs are CD34+ and also express CRHBP, HOPX and LMO2. In an embodiment, the LT-HSCs are Lin-, KIT+, SCA1+, CD150+CD48-CD34-CD135-. In an embodiment, the LT-HSCs are Lin-, KIT+, SCA1+CD150+CD48-CD135-.

### IV. Culture Media Components

The methods of the disclosure utilize a culture media comprising or consisting essentially of specific agonists and/or antagonists of cellular receptors and/or other targets within a signaling pathways, as well as other defined components, as described herein.

In an embodiment, the culture media comprises a TGFß agonist, a bioactive phospholipid, an aryl hydrocarbon receptor (AhR) agonist and a histone deacetylase (HDAC) inhibitor. In embodiments, the culture media further comprises an antioxidant. In embodiments, the culture media further comprises a Notch agonist. In embodiments, the culture media lacks a c-kit ligand and a TPOR agonist.

As used herein, an “agonist” of a cellular receptor or other target within a signaling pathway is intended to refer to an agent that stimulates (upregulates) the cellular receptor or other signaling pathway target. Stimulation of the cellular receptor or other signaling pathway target can be initiated extracellularly, for example by use of an agonist that activates a cell surface receptor involved in the cellular receptor or cellular signaling pathway (e.g., the agonist can be a receptor ligand). Additionally or alternatively, stimulation of cellular signaling can be initiated intracellularly, for example by use of a small molecule agonist that interacts intracellularly with a component(s) of the cellular receptor or signaling pathway.

As used herein, an “antagonist” of a cellular receptor or other target within a signaling pathway is intended to refer to an agent that inhibits (downregulates) the cellular receptor or other signaling pathway target. Inhibition of the cellular signaling pathway can be initiated extracellularly, for example by use of an antagonist that blocks a cell surface receptor involved in the cellular receptor or other signaling pathway target. Additionally or alternatively, inhibition of cellular signaling can be initiated intracellularly, for example by use of a small molecule antagonist that interacts intracellularly with a component(s) of the cellular receptor or signaling pathway.

TGFβ agonists, bioactive phospholipids, aryl hydrocarbon receptor (AhR) agonists, histone deacetylase (HDAC) inhibitors, antioxidants and Notch agonists are known in the art and commercially available. They are used in the culture media at a concentration effective to achieve the desired outcome, e.g., enhancement of HSC engraftment. Non-limiting examples of suitable agonist and antagonist agents, and effective concentration ranges, are described further below.

Agonists of TGFß include agents, molecules, compounds, or substances capable of stimulating (activating) a receptor of the TGFβ family (e.g., by binding to the TGFß family receptor and upregulating its activity) such that the TGFß signaling pathway is stimulated (activated). In an embodiment, the TGFβ agonist is Activin A. In another embodiment, the TGFβ agonist is alantolactone. In an embodiment, the TGFß agonist is Activin A, which is present in the media at a concentration range of 10-30 ng/ml, 15-25 ng/ml or 18-22 ng/ml. In an embodiment, the TGFß agonist is Activin A, which is present in the media in a concentration range of 10-30 ng/ml, 15-25 ng/ml or 18-22 ng/ml, e.g., at a concentration of 20 ng/ml.

In an embodiment, the bioactive phospholipid is lysophosphatidic acid (LPA). In other embodiments, the bioactive phospholipid is sphingosine-1-phosphate (SIP), ceramide-1-phosphate (CIP) or lysophosphatidylcholine (LPC). In an embodiment, the bioactive phospholipid is LPA, which is present in the media at a concentration range of 100-300 μM, 150-250 μM, or 180-220 μM. In an embodiment, the bioactive phospholipid is LPA, which is present in the media at a concentration of 200 μM.

Agonists of AhR include agents, molecules, compounds, or substances capable of stimulating (activating) AhR (e.g., by binding to AhR and upregulating its activity) such that the AhR signaling pathway is stimulated (activated). In an embodiment, the AhR agonist is 6-Formylindolo [3,2-b] carbazole (FICZ). In other embodiments, the AhR agonist is Norisoboldine, Pifithrin-a hydrobromide, MeBIO, ITE or 10-CI-BBQ. In an embodiment, the AhR agonist is FICZ, which is present in the media at a concentration range of 250-750 nM, 400-600 nM or 450-550 nM. In an embodiment, the AhR agonist is FICZ, which is present in the media at a concentration of 500 nM.

In an embodiment, the HDAC inhibitor is valproic acid (VPA). In other embodiments, the HDAC inhibitor is selected from the group consisting of vorinostat, entinostat, Panobinostat, Trichostatin A, mocetinostat, 4-Phenylbutyric acid, ACY-775, GSK3117391, belinostat, romidepsin, MC1568, tubastatin A, Givinostat, dacinostat, CUDC-101, quisinostat, pracinostat, PCI-34051, droxinostat, abexinostat, RGFP966, AR-42, ricolinostat, tacedinaline, fimepinostat, sodium butyrate, curcumin, M344, tubacin, RG2833, resminostat, divalproex sodium, scriptaid, sodium phenylbutyrate, tubastatin A, sinapinic acid, TMP269, CAY10683, TMP195, UF010, tasquinimod, SKLb-23bb, isoguanosine, NKL22, sulforaphane, BRD73594, citarinostat, suberohydroxamic, BRD3308, splitomicin, HPOB, LMK235, Biphenyl-4-sulfonyl chloride, nexturastat A, BML-210, TC-H106, SR-4370, TH34, Tucidinostat, SIS17, parthenolide, wt161, CAY10603, ACY738, Raddeanin A, Tinostamustine, domatinostat, BG45 and ITSA-1. In an embodiment, the HDAC inhibitor is VPA, which is present in the media at a concentration range of 100-200 μM, 125-175 μM or 140-160 μM. In an embodiment, the HDAC inhibitor is VPA, which is present in the media at a concentration of 150 μM.

Antioxidants include agents, molecules, compounds, or substances that prevent or slow the damage to cells caused by free radicals. In an embodiment, the antioxidant is vitamin C. In one embodiment, the vitamin C is L-ascorbic acid phosphate sesquimagnesium salt hydrate, a stable form of vitamin C (Vit. C). In other embodiments, the antioxidant is ascorbic acid, glutathione, ebeselen, N-acetyl-L-cysteine or a-tocopherol. In an embodiment, the antioxidant is vitamin C, which is present in the media at a concentration range of 50-150 μM, 75-125 μM or 90-110 μM. In an embodiment, the antioxidant is vitamin C, which is present in the media at a concentration of 100 μM.

Agonists of Notch include agents, molecules, compounds, or substances capable of stimulating (activating) Notch (e.g., by binding to Notch and upregulating its activity) such that the Notch signaling pathway is stimulated (activated). In an embodiment, the Notch agonist is Yhhu 3792. In other embodiments, the Notch agonist is Jagged 1-2 or DLL1-4. In an embodiment, the Notch agonist is Yhhu 3792, which is present in the media at a concentration range of 500-1000 nM, 600-900 nM or 700-800 nM. In an embodiment, the Notch agonist is Yhhu 3792, which is present in the media at a concentration of 750 nM.

In embodiments, the culture media lacks c-kit ligands. C-kit ligands include agents, molecules, compounds or substances that bind to the c-kit receptor (CD117). In an embodiment, the c-kit ligand is stem cell factor (SCF).

In embodiments, the culture media lacks a TPOR agonist. TPOR agonists include agents, molecules, compounds or substances that agonize the thrombopoietin receptor (TPOR). In an embodiment, the TPOR agonist is thrombopoietin (TPO).

### V. Subjects

Following culture of the HSCs as described herein, the cells are administered to a subject. As used herein, the term “subject” is intended to refer to organisms (e.g., humans, humanized/chimerized animals, and animals) that can serve as recipients of HSCs.

In embodiments, the subject is a human subject. In embodiments, the subject is a vertebrate animal. In embodiments, the vertebrate animal is a mammal. In embodiments, the vertebrate animal is a primate, non-limiting examples of which include chimpanzees, rhesus macaques and cynomolgus macaques. In embodiments, the vertebrate animal is a rodent, non-limiting examples of which include mice and rats. In embodiments, the vertebrate animal is a domesticated animal, non-limiting examples of which include cows, horses, goats, sheep, dogs, cats and chickens.

In other embodiments, the subject is an animal that serves as a non-human animal model, such as an animal that mimics a human disease. The approach of the disclosure can be applied to essentially any animal model known in the art that utilizes HSCs. In embodiments, the animal that serves as a non-human animal model is a rodent, such as a mouse or rat. In embodiments, the animal that serves as a non-human animal model is a primate, such as a chimpanzee, rhesus macaque or cynomolgus macaque. In embodiments, the animal that serves as a non-human animal model has been modified or altered to make it more human-like or similar to another species, i.e., it has been “humanized” or “chimerized.” For example, one or more of the endogenous genes of the animal may be replaced with a human version and/or one or more types of cells within the animal may be replaced with human cells. In embodiments, human CD34+ or CD34-HSCs are administered to the non-human animal model that has already been humanized/chimerized. In embodiments, human CD34+ or CD34-HSCs are administered to the non-human animal model that has not yet been humanized, serving to humanize it. In embodiments, non-human CD34+, CD34-, or CD150+CD41-CD34-Kit+Sca-1+Lineage-HSCs are administered to the non-human animal model (e.g., the species of cells can be matched to the species of the animal model, such as mouse HSCs used in a mouse model). In embodiments, non-human CD34+ or CD34-HSCs that have themselves been humanized (e.g., express one or more human genes) are administered to the non-human animal model.

Cells are administered to the subject by standard methods well established in the art. Sufficient numbers of cells are administered by a suitable route to accomplish the intended purposes, e.g., the intended clinical or research goal and one of ordinary skill in the art can adjust the seeding density of the cells accordingly depending on the cells, the subject and the intended purpose. Non-limiting parameters for administration of cells to a subject are set forth in the Examples.

The present disclosure is further illustrated by the following examples, which should not be construed as further limiting. The contents of figures and all references, patents and published patent applications cited throughout this application are expressly incorporated herein by reference.

### Examples

### Example 1: Primary and Secondary Transplant Methodologies

This example describes the methodologies used for the primary and secondary transplant experiments described in the subsequent Examples.

### Animals and Cells

NSG (NOD.Cg-Prkdcscid Il2rgtm1Wjl) mice purchased from Jackson Laboratory (Bar Harbor, ME) were housed in a specific pathogen-free environment. Human umbilical cord blood (CB) was obtained from the Cleveland Cord Blood Center (Cleveland, OH). Studies were conducted with approval by the Institutional Review Board and Animal Care and Use Committees at Columbia University.

### Culture Media

The recipe for the culture media referred to herein as LT-HSCOPT is shown below in Table 1:

TABLE 1
LT-HSC^{OPT} Culture Media

| Effectors | Role | Concentration | |
|---|---|---|---|
| Activin A | TGFβ agonist | 20 | ng/ml |
| Lysophosphatidic aid (LPA) | Bioactive phospholipid | 200 | nM |
| 6-Formylindolo[3,2-b]carbazole | aryl hydrocarbon | 500 | nM |
| (FICZ) | receptor (AhR) agonist | | |
| Valproic acid (VPA) | Histone deacetylase | 150 | μM |
| | inhibitor | | |

The culture media referred to herein as LT-HSC^{OPT+SCF/TPO} has the same recipe as the LT-HSC^{OPT} culture media plus the addition of stem cell factor (SCF) (10 ng/ml),thrombopoietin (TPO) (100 ng/ml), YHHU-3792 (750 nM) and L-Ascorbic acid 2-phosphate (100 μM). Control media used were basal media (StemSpan™ SFEM II; STEMCELL Technologies) and basal media plus SCF (10 ng/ml) and TPO (100 ng/ml).

### Primary Transplant Studies

Six-week-old female NSG mice were myeloablative conditioned 24 hours prior to the transplantation by irradiation at 1Gy in an X-Ray irradiator (RS-2000, Rad Source Technologies, Inc., Suwanee, GA).

Human cord blood CD34+ cells were thawed, centrifuged at 1300 rpm for 5 min and divided evenly for culture in one of the four different culture media listed for groups B, C, D, and E in Table 2 below. For each media, cells were cultured for 24 h with a starting concentration of 0.624 million cells in 5 ml media.

Twenty-four hours later, each 5 ml cell culture was resuspended in 4 ml of PBS and equal volumes of the resuspended cells were injected into conditioned recipients (0.5 ml resuspended cells per mouse). In this manner, each animal received cells that were derived from the same number of cells in the original lot of frozen Human cord blood CD34+ cells.

An additional group of mice (group A in Table 1) were transplanted with the same lot of freshly thawed human cord blood CD34+ cells (0.5 ml freshly-thawed cells per mouse) that were not cultured in any media prior to transplantation.

TABLE 2
Animal Groups

| | | | | actual CB |
|---|---|---|---|---|
| Groups | Description | N | TBI | HSC |
| | Condition | | 1.0 Gy | per mouse |
| A | Uncultured (Fresh-thawed) | 7 | + | 7.8E+04 |
| B | LT-HSC^{OPT} + SCF/TPO | 8 | + | 5.4E+04 |
| C | LT-HSC^{OPT} | 7 | + | 3.9E+04 |
| D | Basal Media + PS | 8 | + | 4.3E+04 |
| E | SCF + TPO | 8 | + | 4.8E+04 |

### Engraftment Analysis

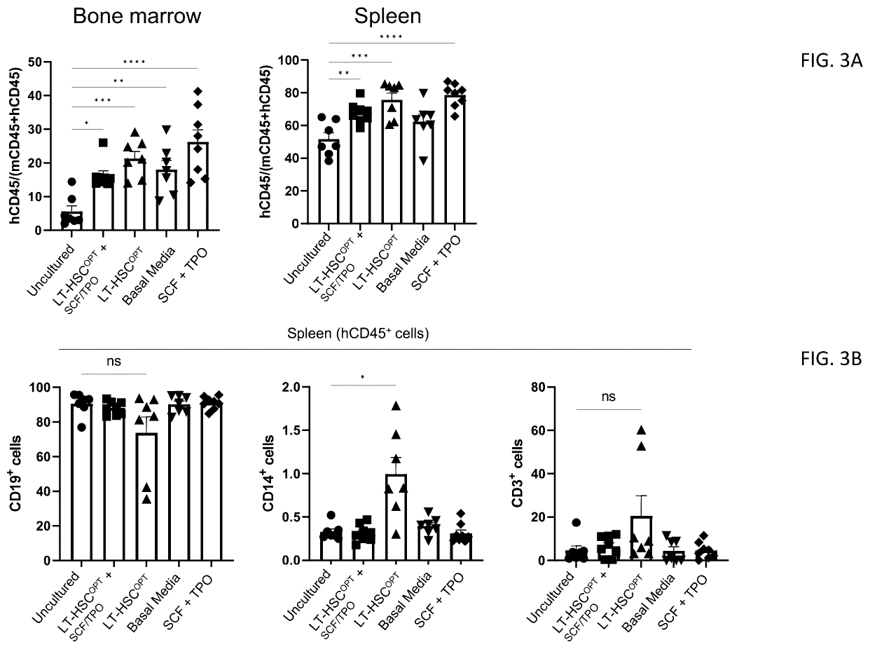

Peripheral blood engraftment was measured by removing blood from the tail veil at different time points and analyzing cells by standard flow cytometry. Blood and spleen cells were stained with antibodies for mCD45, hCD45, CD3, CD8, CD19, CD14 and propidium iodide, a live and dead marker, to exclude dead cells from the analysis. Percentage human engraftment based on hCD45 expression was calculated as: % hCD45+engraftment=hCD45+ cells/(hCD45+ cells+mCD45+ cells)× 100. Ordinary one-way ANOVA with post-hoc Dunnet was used to determine statistical significance. All the groups were compared to control group (uncultured). For data represented over time, two way ANOVA with post hoc Tukey was used to determine statistical significance. For data shown in FIG. 3B, Brown-Forsythe and Welch ANOVA test was used to determine statistical significance (SD across groups were not equal).

Secondary Transplant Studies

20 weeks after primary transplants, femurs and tibia bone marrow cells from primary recipients were harvested. Negative selection using mCD45 (mouse hematopoietic cells) and hCD3 (human T-cells) microbeads from Miltenyi Biotec was performed. After that cells of interest were separated using LS columns. Lineage, hCD45 and CD34 levels was analyzed by FACS. NSG mice were exposed to total body irradiation at 1.5gy before cell injection. 2.4 million of LinhCD45+CD34+ cells were injected per mice. Peripheral blood engraftment was measured by removing blood from tail vein and analyzing by flow cytometry.

### Example 2: Flow Cytometry Validation of Culture Media for Increased Expression of LT-HSC Markers

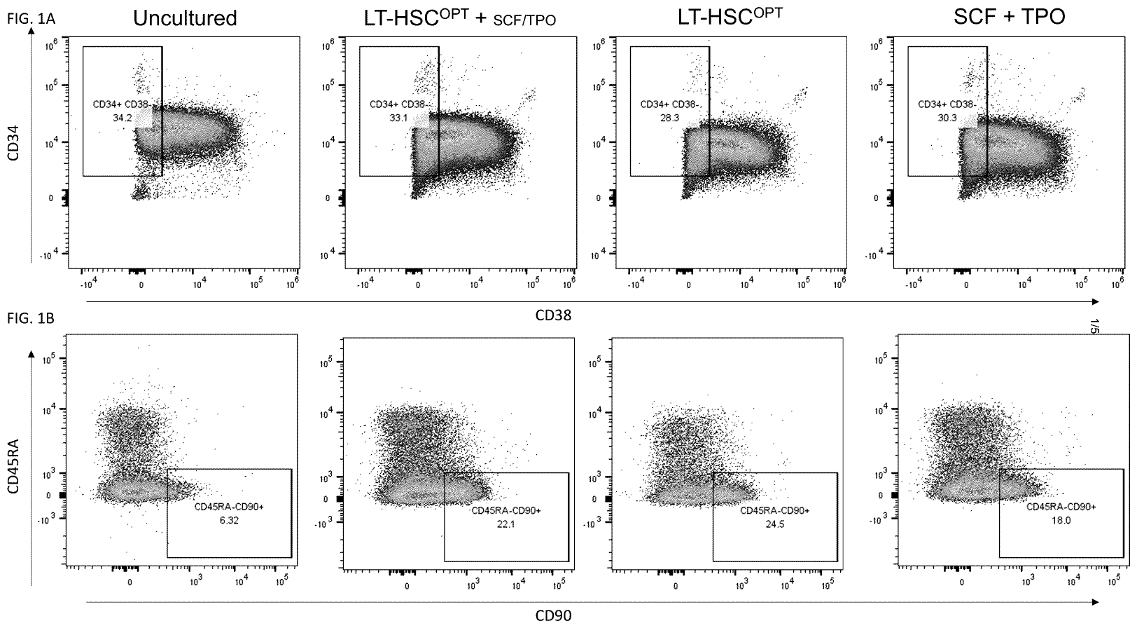

In this example, human cord blood CD34+ cells were cultured for 24 hours in the culture media described in Example 1 and flow cytometry analysis was used to assess markers of the LT-HSC state to thereby validate the HSC phenotype after 24 hours of culture. Markers tested included Lineage (CD3, CD14, CD16, CD19, CD20, CD56), CD34, CD38, CD45RA and CD90. Additionally, cells were grown in media with SCF and TPO as a control, commonly used in the art to promote HSC expansion. The immunophenotype of LT-HSC, namely Lin CD34⁺CD38-CD45RA CD90⁺was evaluated. The results for CD34/CD38 staining are shown in FIG. 1A and the results for Lin-CD34⁺CD38-CD45RA/CD90 staining are shown in FIG. 1B. Flow cytometry analysis showed comparable levels of CD34 and CD38 expression in all conditions tested. On the other hand, an increase in the CD45RA CD90+ population as observed upon culturing conditions, in which LT-HSC^{OPT} increased almost 4 times the percentage of cells that are CD45RA-CD90⁺, suggesting an increase in HSC activity when compared to uncultured conditions.

### Example 3: Culture in LT-HSC^{OPT} Media Increases Long Term Peripheral Blood Engraftment of Cord Blood CD34 Cells as Compared to Uncultured Conditions

In this example, primary transplant studies were carried out as described in Example 1 and the effect of the different culture media on long term peripheral blood engraftment was examined.

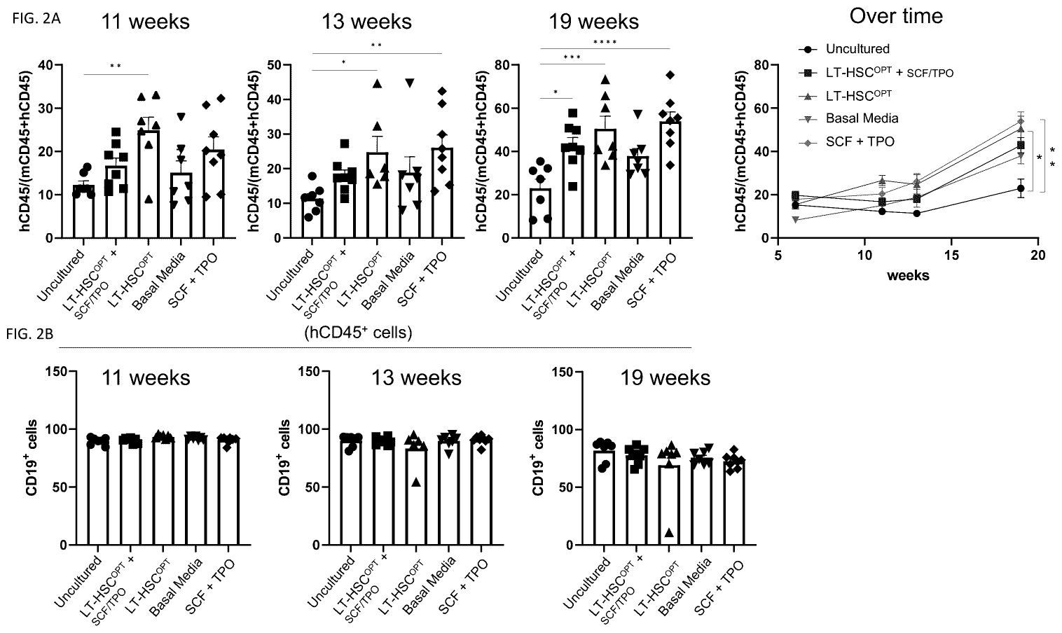

The standard assay established in the art to measure HSC function involves injection of HSCs in irradiated mice and monitoring the functional contribution of these cells to blood generation in vivo. To assess whether HSC activity was enhanced by 24 hours of culture with the LT-HSC culture media described herein, the progeny of 50,000 CB CD34⁺ cells were transplanted into sub-lethally irradiated immunodeficient NOD-SCID IL-2rynull (NSG) mice. In a clinical setting, HSC transplant into patients is performed after thawing the cord blood unit without any culture condition. To mimic clinical conditions, cells were also transplanted immediately after thawing (also referred to herein as uncultured). CD34⁺engraftment in the peripheral blood of the NSG mice was evaluated by collecting blood from the tail vein at different time points. Representative results are shown in FIG. 2A (hCD45 cells) and FIG. 2B (CD19⁺B cells). The results demonstrated that at 11, 13 and 19 weeks after injection, culture for 24 hours in LT-HSC^{OPT} media improved engraftment of cord blood cells as compared to uncultured cells. SCF and TPO condition also increased engraftment when compared to uncultured cells. Flow analysis of hCD45 cells showed that most human cells found on peripheral blood (80-90%) differentiated to CD19⁺B-cells.

### Example 4: Culture in LT-HSC^{OPT} Media Increases Bone Marrow and Spleen Engraftment of Cord Blood CD34 Cells as Compared to Uncultured Conditions

In this example, the long term engraftment (19 weeks) of cord blood CD34⁺ cells in spleen and bone marrow was examined from the same primary transplant studies described in Examples 1 and 3. At 19 weeks after cell transplantation, animals were sacrificed and single cells from bone marrow and spleen were analyzed by flow cytometry. Representative results are shown in FIG. 3A (hCD45 cells) and FIG. 3B (hCD19⁺, CD14⁺ and CD3⁺ cells). Human CD45⁺ cells were detected on spleen and bone marrow 19 weeks after cord blood transplantation. Both SCF+TPO and LT-HSC^{OPT} media increased engraftment of human cells in the spleen and bone marrow. Human CD45⁺ cells found in spleen and bone marrow were comprised of multiple hematopoietic lineages, with B-cells representing most of these cells (FIG. 3B). Additionally, a significant increase in CD14⁺myeloid cells was observed in animals that received cells treated with LT-HSC^{OPT} media (FIG. 3B).

### Example 5: Use of LT-HSC^{OPT} Media Increases Human Cell Engraftment in a Secondary

### Transplant Model

To further evaluate the repopulating potential of cells cultured in LT-HSC^{OPT} media, secondary transplants of cells harvested from bone marrow from primary recipients were performed, the results of which are described in this example.

In the past, there was a consensus in the field that 16 weeks after transplants was sufficient to study the long-term output of HSC. Nowadays, secondary transplant is the gold standard assay to measure HSC activity (retention of self-renewal capacity). The secondary transplantation forces HSC to exit from dormancy, due to proliferative pressure. This way, only cells with strong self-renewal capacity can sustain their output.

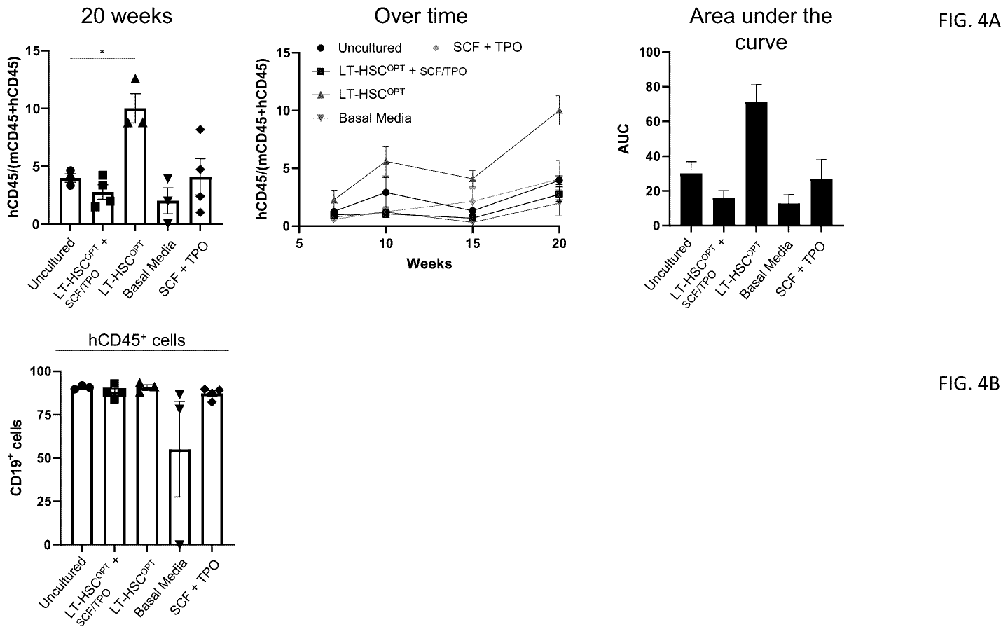

Secondary transplants of cells harvested from bone marrow from primary recipients were performed as described in Example 1. Pooled bone marrow cells were negatively selected for mouse CD45⁺ cells as well as human CD34⁺ cells. After selection, 2.4 million CD34⁺ cells per mice were injected into secondary recipients. Reconstitution was monitored for 20 weeks post-transplantation. Peripheral blood engraftment was analyzed by flow cytometry as described in Example 1. Results are shown in FIG. 4A (hCD45 cells) and FIG. 4B (CD19⁺B cells). The results demonstrated that peripheral blood engraftment was enhanced in mice that received cells originally cultured in LT-HSC^{OPT}, suggesting higher HSC activity under this condition. Analysis of area under the curve confirmed that LT-HSC^{OPT} media improved significantly secondary engraftment when compared to any of the other conditions (FIG. 4A). Again, flow cytometry analysis of hCD45 cells showed that the majority of human cells found in peripheral blood (80-90%) differentiated to CD19⁺B-cells (FIG. 4B).

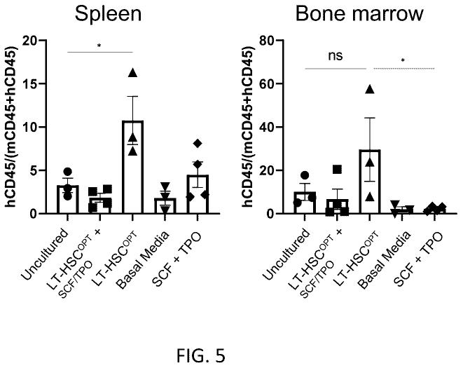

Spleen and bone marrow secondary engraftment was also analyzed by flow cytometry. Single cells from bone marrow and spleen were harvested after 20 weeks, and the presence of human antigens was measured. Results are shown in FIG. 5. The results showed that both spleen and bone marrow engraftment was enhanced in mice that received cells originally cultured in LT-HSC^{OPT}, confirming higher HSC activity under this condition. Spleen engraftment was more than double using LT-HSC^{OPT} media pre-culture as compared to uncultured cells. Bone marrow engraftment was more than double as well; however, not statistically significant due to high variation across animals.

Altogether these data shows that culture of CD34 cells for 24 hours in LT-HSC^{OPT} media prior to transplantation improved (enhanced) primary long term (enduring) transplant as well as secondary long term (enduring) transplant as compared to uncultured cells, confirming higher HSC activity in cells cultured with LT-HSC^{OPT} media prior to transplantation.

### EQUIVALENTS

Those skilled in the art will recognize, or be able to ascertain using no more than routine experimentation, many equivalents of the specific embodiments of the invention described herein. Such equivalents are intended to be encompassed by the following claims:

1. A method of preparing hematopoietic stem cells (HSCs) for transplantation into a subject, the method comprising:
culturing HSCs in a culture media comprising a TGFb agonist, a bioactive phospholipid, an aryl hydrocarbon receptor (AhR) agonist and a histone deacetylase (HDAC) inhibitor for 12-36 hours, wherein the culture media lacks stem cell factor (SCF) and thrombopoietin (TPO); and
administering the HSCs into the subject.

2. The method of claim 1, wherein the HSCs are human CD34+HSCs.

3. The method of claim 1, wherein the HSCs are human CD34-HSCs.

4. The method of claim 1, wherein the HSCs are non-human CD34+, CD34-, or CD150+CD41-CD34-Kit+Sca-1⁺Lineage-HSCs.

5. The method of claim 4, wherein the HSCs are primate HSCs.

6. The method of claim 4, wherein the HSCs are rodent HSCs.

7. The method of claim 1, wherein the HSCs have been humanized or chimerized.

8. The method of claim 1, wherein the HSCs are cultured in the culture media for 18-32 hours.

9. The method of claim 1, wherein the HSCs are cultured in the culture media for about 24 hours.

10. The method of claim 1, wherein the HSCs are cultured in the culture media for 24 hours.

11. The method of claim 1, wherein culturing the HSCs enhances engraftment of the HSCs in the subject as compared to absence of culturing prior to administration.

12. The method of claim 11, wherein enhanced engraftment comprises increased engraftment of the HSCs in the peripheral blood.

13. The method of claim 11, wherein enhanced engraftment comprises increased engraftment of the HSCs in the spleen and/or bone marrow.

14. The method of claim 1, wherein the culture media consists essentially of a basal media, a TGFb agonist, a bioactive phospholipid, an AhR agonist and an HDAC inhibitor.

15. The method of claim 1, wherein the TGFb agonist is Activin A.

16. The method of claim 1, wherein the TGFb agonist is alantolactone.

17. The method of claim 1, wherein the bioactive phospholipid is lysophosphatidic acid (LPA).

18. The method of claim 1, wherein the bioactive phospholipid is sphingosine-1-phosphage (S1P), ceramide-1-phosphate (CIP) or lysophosphatidylcholine (LPC).

19. The method of claim 1, wherein the AhR agonist is 6-Formylindolo [3,2-b] carbazole (FICZ).

20. The method of claim 1, wherein the AhR agonist is Norisoboldine, Pifithrin-a hydrobromide, MeBIO, ITE or 10-CI-BBQ.

21. The method of claim 1, wherein the HDAC inhibitor is valproic acid (VPA).

22. The method of claim 1, wherein the HDAC inhibitor is selected from the group consisting of vorinostat, entinostat, Panobinostat, Trichostatin A, mocetinostat, 4-Phenylbutyric acid, ACY-775, GSK3117391, belinostat, romidepsin, MC1568, tubastatin A, Givinostat, dacinostat, CUDC-101, quisinostat, pracinostat, PCI-34051, droxinostat, abexinostat, RGFP966, AR-42, ricolinostat, tacedinaline, fimepinostat, sodium butyrate, curcumin, M344, tubacin, RG2833, resminostat, divalproex sodium, scriptaid, sodium phenylbutyrate, tubastatin A, sinapinic acid, TMP269, CAY10683, TMP195, UF010, tasquinimod, SKLb-23bb, isoguanosine, NKL22, sulforaphane, BRD73594, citarinostat, suberohydroxamic, BRD3308, splitomicin, HPOB, LMK235, Biphenyl-4-sulfonyl chloride, nexturastat A, BML-210, TC-H106, SR-4370, TH34, Tucidinostat, SIS17, parthenolide, wt161,CAY10603, ACY738, Raddeanin A, Tinostamustine, domatinostat, BG45 and ITSA-1.

23. The method of claim 1, wherein the TGFb agonist is Activin A, bioactive phospholipid is LPA, the AhR agonist is FICZ and the HDAC inhibitor is VPA.

24. The method of claim 23, wherein Activin A is present in a concentration range of 15-25 ng/ml, LPA is present at in a concentration range of 150-250 nM, FICZ is present in a concentration range of 400-600 nM and VPA is present in a concentration range of 100-200 μM.

25. The method of claim 23, wherein Activin A is present at a concentration of 20 ng/ml, LPA is present at a concentration of 200 nM, FICZ is present at a concentration of 500 nM and VPA is present at a concentration of 150 μM.

26. The method of claim 1, wherein the HSCs are human, non-human, or humanized/chimerized HSCs from umbilical cord blood.

27. The method of claim 1, wherein the HSCs are human, non-human, or humanized/chimerized HSCs from bone marrow.

28. The method of claim 1, wherein the HSCs are human, non-human, or humanized/chimerized HSCs from peripheral blood.

29. The method of claim 1, wherein the HSCs are human, non-human, or humanized/chimerized HSCs from fetal liver.

30. The method of claim 1, wherein the subject is human.

31. The method of claim 1, wherein the subject is a non-human animal.

32. The method of claim 1, wherein the subject has been humanized/chimerized.

33. A method of enhancing engraftment of hematopoietic stem cells (HSCs) in a subject, the method comprising:
culturing HSCs in a culture media consisting essentially of a basal media, a TGFb agonist, a bioactive phospholipid, an aryl hydrocarbon receptor (AhR) agonist and a histone deacetylase (HDAC) inhibitor for 12-36 hours; and
transplanting the HSCs into the subject,
wherein engraftment is enhanced compared to not culturing the HSCs prior to transplanting.

34. The method of claim 33, wherein the HSCs are human CD34+HSCs.

35. The method of claim 33, wherein the HSCs are human CD34-HSCs.

36. The method of claim 33, wherein the HSCs are non-human CD34+, CD34-, or CD150+CD41-CD34-Kit+Sca-1+Lineage-HSCs.

37. The method of claim 36, wherein the HSCs are primate HSCs.

38. The method of claim 36, wherein the HSCs are rodent HSCs.

39. The method of claim 33, wherein the HSCs have been humanized/chimerized.

40. The method of claim 33, wherein the HSCs are cultured in the culture media for 18-32 hours.

41. The method of claim 33, wherein the HSCs are cultured in the culture media for about 24 hours.

42. The method of claim 33, wherein the HSCs are cultured in the culture media for 24 hours.

43. The method of claim 33, wherein enhanced engraftment comprises increased engraftment of the HSCs in the peripheral blood.

44. The method of claim 33, wherein enhanced engraftment comprises increased engraftment of the HSCs in the spleen and/or bone marrow.

45. The method of claim 33, wherein the TGFb agonist is Activin A, the bioactive phospholipid is LPA, the AhR agonist is FICZ and the HDAC inhibitor is VPA.

46. The method of claim 45, wherein Activin A is present in a concentration range of 15-25 ng/ml, LPA is present at in a concentration range of 150-250 nM, FICZ is present in a concentration range of 400-600 nM and VPA is present in a concentration range of 100-200 μM.

47. The method of claim 45, wherein Activin A is present at a concentration of 20 ng/ml, LPA is present at a concentration of 200 nM, FICZ is present at a concentration of 500 nM and VPA is present at a concentration of 150 μM.

48. The method of claim 33, wherein the HSCs are from umbilical cord blood.

49. The method of claim 33, wherein the HSCs are from bone marrow.

50. The method of claim 33, wherein the HSCs are from peripheral blood.

51. The method of claim 33, wherein the subject is human.

52. The method of claim 33, wherein the subject is a non-human animal.

53. The method of claim 33, wherein the subject has been humanized/chimerized.
